(19) United States
(12) Patent Application Publication
(10) Pub. No.: US 2025/0188009 A1
(43) Pub. Date: Jun. 12, 2025

(54) OPTIMIZED CATALYST PROCESSING AND RECYCLING IN THE SYNTHESIS OF METHACROLEIN

(71) Applicant: Röhm GmbH, Darmstadt (DE)

(72) Inventors: Rudolf Burghardt, Darmstadt (DE); Florian Zschunke, Frankfurt (DE); Steffen Krill, Muehltal (DE); Torsten Panak, Stockstadt/Rhein (DE); Eduard Rundal, Frankfurt (DE); Gerhard Kölbl, Gernsheim (DE)

(73) Assignee: Röhm GmbH, Darmstadt (DE)

(21) Appl. No.: 18/689,139

(22) PCT Filed: Sep. 2, 2022
(86) PCT No.: PCT/EP2022/074428

§ 371 Date: Mar. 5, 2024

(30) Foreign Application Priority Data
Sep. 6, 2021 (EP) 21194990.4

(51) Int. Cl. C07C 45/45; C07C 45/82

(52) U.S. Cl. CPC C07C 45/45; C07C 45/82

(57) ABSTRACT

A process and a production plant for producing methacrolein from formaldehyde and propionaldehyde in the presence of a homogeneous catalyst mixture which is based at least on an acid and a base can be used. A catalyst mixture commonly used for this synthesis has an organic acid and a secondary amine. The presence of the homogeneous catalyst mixture in the reaction mixture is an essential prerequisite for achieving a high yield. The process realizes better water management, thus making it possible not only to reduce the total catalyst amount required but also to reduce undesired catalyst byproducts more efficiently than before.

### CROSS-REFERENCE TO RELATED APPLICATIONS

This application is a National Stage entry under § 371 of International Application No. PCT/EP2022/074428, filed on Sep. 2, 2022, and which claims the benefit of priority to European Patent Application No. 21194990.4, filed on Sep. 6, 2021. The content of each of these applications is hereby incorporated by reference in its entirety.

### BACKGROUND OF THE INVENTION

### Field of the Invention

The present invention relates to a process and a production plant for producing methacrolein from formaldehyde and propionaldehyde in the presence of a homogeneous catalyst mixture which is based at least on an acid and a base. A catalyst mixture commonly used for this synthesis consists of an organic acid and a secondary amine. The presence of the homogeneous catalyst mixture in the reaction mixture is an essential prerequisite for achieving a high yield.

The reaction and all processes for producing methacrolein from propionaldehyde and formaldehyde afford not only the desired target product methacrolein but also a liquid aqueous phase in which the homogeneous catalyst mixture is present in dissolved form. This aqueous phase is partly recycled in the process and partly disposed of in a complex and costly manner as highly contaminated wastewater, for example in an incineration stage. The employed homogeneous catalyst mixture is lost with the disposal of the wastewater. It cannot therefore be reused for the reaction and must be procured again at considerable financial cost. In addition the use of certain secondary amines as part of the acid/base catalyst mixture forms undesired downstream products such as for example trimethylamine which reduce the activity of the catalyst mixture, can lead to undesired further secondary reactions such as for example anionically-induced polymerization of methacrolein and bind the acid proportion of the catalyst mixture as salt.

### Description of Related Art

A process for producing methacrolein based on propanal and formaldehyde for later conversion into MMA is described in WO 2016/042000A1 for example. This methacrolein process is performed at a feed temperature between 100° C. and 150° C. and a maximum temperature of 180° C. in the output of the tubular or plate reactor. The water content is stated as a value between 45% and 85% and the ratio of amine to propionaldehyde in the reactor feed is indicated as greater than 5 mol %. The pressure in the reactor is in turn greater than the boiling pressure and the residence time is between 1 and 30 sec. The catalyst employed is especially a combination of dimethylamine and acetic acid. The yields are about 98.3% at conversions of about 99.9%. The molar ratio of dimethylamine to propionaldehyde in the feed to the reactor is 2.5 mol %.

EP 3 786 146 describes a workup process for the reactor output. The reactor output is decompressed in a flash vessel and the methacrolein-containing gaseous phase is condensed in a condenser. The methacrolein phase and an aqueous phase are subsequently formed in a decanter. The aqueous “flash phase” is passed into a stripping column and freed of residual amounts of methacrolein. The vapours from this column pass into the condenser and are likewise collected as a biphasic system in the abovementioned decanter. The aqueous phase at the bottom of the methacrolein column is partially recycled to the reactor and the remainder discharged as wastewater. This wastewater is optionally concentrated using membrane processes and a catalyst-rich phase recycled back to the reactor. The aqueous reflux may optionally be recycled to the wastewater stripping column. This aqueous phase may optionally also be depleted of methacrolein in a further distillation column and discharged as wastewater.

DE 3213681 A1 describes the production of methacrolein from propionaldehyde and formalin at reaction temperatures of 150° C. to 300° C. in the liquid phase. The reaction time is up to 25 min. The employed catalyst is likewise a combination of dimethylamine and acetic acid.

U.S. Pat. No. 4,408,079 in turn describes the same reaction at markedly lower temperatures of 0° C. to 150° C. in the presence of secondary amine and carboxylic acid, dicarboxylic acid and polycarboxylic acid. Examples with a catalyst system composed of diethanolamine and oxalic acid show yields of about 94.3%. However, the processes with dimethylamine generally appear to provide higher yields at higher temperatures. These also have the advantage that the reaction need not be performed in stirred tank reactors that are costly and complex to operate.

Especially the recycling of the homogeneous catalyst composed of secondary amine and organic acid is a much-discussed aspect of the prior art.

DE 3213682 A1, cited above, also discloses a catalyst recycling: Thus at relatively high amine concentration in the bottoms outflow, the water may be partially distillatively removed and the catalyst solution recycled back into the reactor. The water content in the catalyst solution is not disclosed and the possible separation of trimethylamines formed as a byproduct is also missing here.

U.S. Pat. No. 4,408,079 describes the recycling of the employed diethanolamine in combination with sulfuric acid. Methacrolein is here produced at 40° C. and a stirring time of 2 h for example. After the distillative removal of the methacrolein the remaining water phase continues to be used as catalyst. The catalyst solution is regenerated only by removal of water. The discharging of further reaction products is not mentioned.

In the synthesis of methacrolein with diethanolamine and oxalic acid, disclosed in the same document, the reaction is carried out for example at about 40° C. to 50° C. Water introduced with the formaldehyde and the water of reaction is distillatively removed in a second step after distillative removal of methacrolein to afford a catalyst solution which may be reused. Reaction and recycling are performed until the organic product has a methacrolein content of less than 90% by weight, and only then is the catalyst system replaced. Accordingly the purity of the target product methacrolein in this process cannot be maintained at a constant level/above 90% by weight possibly as a result of a falling propionaldehyde conversion and enrichment of propionaldehyde in the tops fraction or possibly as a result of enrichment of undesired byproducts in the tops fraction. This process may thus suffer from excessively low catalyst dosing to the reactor since the propionaldehyde conversion apparently cannot be maintained at a constantly high level as a result of insufficient dosing of fresh catalyst or loss of active catalyst.

This corresponds to a molar yield of methacrolein of 63.4 mol of methacrolein based on 1 mol of secondary amine or 0.0156 mol of diethanolamine per mol of methacrolein or 1.65 g of diethanolamine per mol of methacrolein.

The process according to EP 3 786 146 employs 0.025 mol of dimethylamine based on 1 mol of propionaldehyde. A yield of 98.3% is described and the specific demand for dimethylamine per mol of methacrolein is 1.14 g of DMA/mol of methacrolein. The specific usage amount for the amine is thus markedly lower here and since dimethylamine is much less costly than diethanolamine the process with dimethylamine is markedly more economic. The advantage remains even if no workup of the wastewater and recovery of the catalyst is carried out.

The abovementioned examples have all been performed in a batch process mode. U.S. Pat. No. 4,408,079 also describes a continuous process. Propionaldehyde and formaldehyde are then converted into methacrolein in a stirred tank cascade composed of two tanks at about 50° C. and a residence time of 1.27 h. The catalyst system employed was a 60% solution of diethanolamine and oxalic acid in a molar ratio of 2:1. Diethanolamine is employed equimolarly to propionaldehyde and the methacrolein yield was 94.2%. The catalyst mixture was subsequently concentrated to the original nitrogen content and presumably hydrogen content and recycled.

The process disclosed in U.S. Pat. No. 4,408,079 has crucial disadvantages:
1) The higher catalyst consumption despite recycling.
2) The significantly lower conversion of the propionaldehyde as a consequence of high backmixing in the stirred tanks.
3) The markedly higher capital and maintenance costs for the stirred tank reactors compared to a flow tube.
4) Only water is discharged in the catalyst concentration. Byproducts formed by reaction of the secondary amine and high-boilers are apparently left behind.

The teaching of U.S. Pat. No. 4,408,079 also comprises an aspect that the use of low molecular weight amines results in an amine-contaminated methacrolein that requires costly and complex workup before further use. Losses of amines are also to be expected in the concentration process which follows the separation of methacrolein. These increase with the volatility of the amines. Therefore, according to the teaching of U.S. Pat. No. 4,408,079 preference is given to secondary amines having a boiling point of more than 130° C. Dimethylamine has a boiling point of 3° C. and therefore a catalyst workup of a dimethylamine-containing solution by evaporation/distillation contradicts this teaching.

A wide variety of literature citations, in particular WO 2015/065610, WO 2018/217961, WO 2018/217962, WO 2018/217963 and WO 2018/217964, describe a process for producing water-free and methanol-free methacrolein and the further use thereof for producing methacrylic acid and MMA. The employed catalyst is a mixture of dimethylamine and acetic acid. The reaction mixture is passed into a phase separator at temperatures below 20° C. and separated therein. The aqueous phase is supplied to a column. The tops stream of this column which contains especially methacrolein and methanol is worked up in a different fashion. The column further comprises a side draw where a phase comprising a very large proportion of water is withdrawn. The bottoms product is discharged to an extent of 10% to 25% and recycled to the reactor to an extent of 75% to 90%. What is concerned here is thus a combination of separating organic components such as methacrolein or methanol and concentrating catalyst. However, recycling without concentration has not hitherto been described.

The water contents in the concentrate are at most 85% by weight. Nothing is said about any remaining trimethylamine (TMA). This byproduct must inevitably be formed in the reactor under the described reaction conditions. Since discharging of TMA is not mentioned it must be assumed that TMA accumulates in the system. If TMA is discharged it is highly probable that TMA passes into the tops product with methacrolein. This could result in extremely undesirable secondary reactions during further processing of the methacrolein. It is known that bases undergo highly exothermic reaction with methacrolein in the alkaline range.

The disadvantage of this process is thus the catalyst concentration and the separation of the methacrolein in one stage. Separation of TMA overhead would be expected to result in reaction of TMA with methacrolein which can be very exothermic, similarly to methylol synthesis.

A further disadvantage of this process is that the greatly excessive water content in the concentration stage does not allow trimethylamine to be discharged. This also has the result that more acetic acid is bound.

A further disadvantage of this process is that the reactor output is completely cooled down from temperatures of at least 160° C. to about 20° C. and the obtained liquid phases must then be re-heated to boiling temperature in the respective distillation stage. This is energetically unfavourable.

EP 2 883 859 describes a methacrolein synthesis employing mixtures of dimethylamine and trimethylamine. A ratio of secondary amine to tertiary amine between 20 to 1 and 1 to 3 is claimed. Surprisingly, and contrary to the teaching in the further prior art, TMA here has a rather catalytic effect on MAL synthesis although a sufficiently high concentration of DMA is always necessary to achieve the desired high conversion and selectivity. EP 2 883 859 also describes possible recycling processes which are especially based on downstream membrane separation stages: The wastewater may be still further separated in this case via an additional distillation prior to the incineration in order thus to return this for example concomitantly discharged product to the reaction circuit or the product workup. The retentate of the membrane stage may be wholly or partially recycled into the reactor or the workup. It is especially preferable when the aqueous phase of the reaction is discharged and separated into two phases via a membrane. The obtained amine-containing wastewater is then alternatively disposed of, for example in a biological workup, and the retentate of the membrane separation is partially recycled into the reactor. It is also possible to effect further concentration of the retentate in a distillation stage. The obtained concentrate may then be partially passed into the reactor.

Also described here and alternatively to membrane separation the aqueous phase of the reaction may be discharged and separated in a distillation. The distillation bottoms thus obtained are then recycled into the reactor.

Various concentration processes/the combination thereof in the application of dimethylamine as catalysts are/is described in the prior art. What all of these processes have in common is that they do not sufficiently take into account the problem of TMA that is formed and that this either disadvantageously accumulates in the plant or is removed using a great deal of energy. The present prior art leaves a great deal of potential for process improvements.

The membrane process has the disadvantage that it does not allow separation of trimethylamine. The accumulation of TMA results in the abovementioned problems so that at most only a markedly reduced degree of recycling can be achieved. In addition the aqueous phase must initially be cooled to about 20° C. to 40° C. which is relatively unfavourable from an energetic standpoint.

In summary it is noted that according to the prior art a synthesis at elevated temperatures and pressures using dimethylamine and acetic acid represents a favoured production process for methacrolein. These processes in particular have the feature that they are performable with particularly high yields and a comparatively low consumption of secondary amine and acid. However, despite the relatively low consumption of catalyst of for example about 0.025 mol of dimethylamine per mol of propionaldehyde and about 0.0275 mol of acetic acid per mol of propionaldehyde considerable amounts of catalysts are still consumed.

Furthermore the prior art discusses the simultaneous formation of trimethylamine as a byproduct when using dimethylamine in the production of methacrolein. Although trimethylamine has a certain catalytic activity it is markedly less active and less selective compared to dimethylamine. As a base trimethylamine binds acetic acid as a salt in a molar ratio of 1:1 and the acid bound in the form of the salt can therefore exhibit only markedly reduced catalyst activity. In addition, trimethylamine can in some cases induce unwanted secondary reactions that adversely affect the yield and operating time of a methacrolein production process.

### SUMMARY OF THE INVENTION

### Problem

A problem addressed by the present invention was accordingly that of providing an economic process for producing methacrolein from propionaldehyde which especially features a high yield and low catalyst consumption.

A problem addressed by the present invention was especially that of significantly reducing the consumption of dimethylamine and the acid component such as for example acetic acid in the production of methacrolein and thus also markedly optimizing the production of methacrolein from C2 components in terms of economy and sustainability.

A problem addressed by the present invention was moreover that of developing a production process for methacrolein which allows separation of trimethylamine. A problem addressed by the present invention was thus implicitly that of performing the process such that in the production of methacrolein fewer secondary reactions occur and the demand for acetic acid/the acid component is further reduced.

A problem addressed by the present invention was moreover that of developing a production process which, in addition to addressing the above problems, may be performed in an energy efficient fashion. This further increases the economy and sustainability of the process.

Further problems that are not mentioned explicitly may be apparent from the description of the invention that follows, without being set out explicitly herein.

### Solution

These problems are solved by providing a novel process for continuously performing a Mannich reaction in which methacrolein is produced in a reactor I from formaldehyde and propionaldehyde with at least one acid and dimethylamine as catalysts. This novel process especially has the feature that the output of reactor I is passed directly or indirectly into a distillation column I in which a methacrolein-containing low-boiling phase is separated from an aqueous high-boiling phase having a water content of greater than 85% by weight.

It is a further feature of the present process that this aqueous high-boiling phase is partially directly or indirectly passed into a distillation column II from which the gaseous tops stream is directly or indirectly, wholly or partially, sent to a thermal oxidizer. According to the invention the likewise obtained liquid bottoms phase from this distillation column II has the feature that compared to the high-boiling aqueous phase of distillation column I it has a water content which is at least 20% by weight, preferably at least 25% by weight and very particularly between 40% by weight and 55% by weight, lower. According to the invention this liquid bottoms phase is directly or indirectly, wholly or partially, recycled into reactor I.

For clarity it is noted that the formulation “at least 20% by weight lower” is presently to be understood as referring to an absolute comparison of values. This is to be understood as meaning that a value at least 20% by weight lower than a water content of 85% by weight has a maximum value of 65% by weight in the context of the present invention.

Distillation column II may be a column comprising random packings or structured packings or trays of any desired design. Different operating modes of these columns are conceivable. The feed is for example introduced to the top of the column above the packing and the gas stream withdrawn at the column top. The aqueous bottoms phase may then be withdrawn at the column bottom. The gas stream is generally sent directly to a thermal oxidizer. This is especially advantageous since residual amounts of methacrolein in the gas stream after the condensation can in some cases still lead to an undesired polymerization in the alkaline phase.

The column may be operated such that the feed of the column is run into the middle of the column or at another dosing point of the packing between the column top and column bottom and an aqueous reflux is recycled at the column top above the packing. However, this presupposes a partial or complete condensation of the tops product. This procedure may make it possible to achieve a slightly cleaner tops product and a higher retention of acetic acid.

As a further alternative for concentration it is also possible to perform a single-stage or multi-stage evaporation without structured or random packings. However, this may reduce retentions of acetic acid. Nevertheless a simple evaporator stage entails markedly lower capital costs compared to a distillation column, which would in turn be highly advantageous. Contemplated evaporators include shell and tube evaporators, plate evaporators and thin film evaporators. These can be operated with natural liquid circulation and forced circulation.

The concentration of the catalyst requires energy which is generally provided as steam. Also conceivable is a multi-stage distillation with subsequent vapour compression and utilization of the compressed vapours in the respective subsequent distillation stage. This setup enables significant steam savings. Generally a maximum of three stages makes economic sense. The condensate may then finally also be sent for disposal as wastewater in a biological wastewater treatment plant. Concentration of the condensate by reverse osmosis is also conceivable. The concentrate is then incinerated and the permeate sent to a biological wastewater treatment. One approach for achieving high retentions is to first adjust the condensate to neutral or weakly acidic pH with acetic acid. The trimethylamine salts have very high retention rates. This is feasible especially if the saving through reduced energy consumption is markedly greater than the cost of the additional acetic acid.

In the present process it is preferable when the methacrolein-containing low-boiling phase of distillation column I is condensed in a condenser I connected downstream of distillation column I. It has proven particularly advantageous when this condensate from condenser I is separated into a liquid aqueous phase and a liquid methacrolein-rich phase in a downstream phase separator I.

The condensers are generally shell and tube heat exchangers in which the product to be condensed is run in the tube and the cooling medium is run in the shell. The condensation generally employs cooling water at about 20° C. to 40° C. In the condensation of polymerizable substances such as for example methacrolein the top of the condensers should be sprayed with an aqueous stabilizer solution such as for example TEMPOL solution (1% to 10% by weight) so that the surface of the tubes at which polymerizable substances such as methacrolein condense are wetted with stabilizer and polymerizable condensates formed are in contact with the stabilizers. It is moreover possible to employ a plurality of condensers connected in series with falling coolant flow temperatures to achieve condensation that is as complete as possible. For example the first condenser may be operated with cooling water at about 20° C. and the second condenser with cooling brine at about 4° C. The offgas from the condensers may be sent to an incineration or offgas scrubbing.

Phase separators are generally horizontal containers which depending on the requirements in terms of desired separation sharpness of the liquid-liquid separation may also be fitted with separation aids such as coalescing aids or filter media. In such apparatuses the light phase is generally withdrawn in the upper zone and the heavy phase withdrawn from the container lower down. Control of the phase interface may generally be performed by measurement using a sensor. The position of the phase interface is controlled by altering withdrawal of the aqueous phase. The organic phase drains freely. A further option is for a chamber for the organic phase to also be connected in the phase separator. The organic phase then flows into this chamber over a weir. Pressure equalization is typically also effected in the phase separator via a vent conduit. Normally, this vent conduit is connected to an incineration or offgas scrubbing.

Independently or in addition it has proven particularly advantageous when the reactor output from reactor I is first decompressed and passed into a flash vessel. In this flash vessel a methacrolein-rich gas phase is separated from a high-boiling aqueous liquid phase, wherein this liquid phase may be discharged from the flash vessel into distillation column I.

The advantage of the flash vessel is a separation of a large part of the gaseous product before the feed is passed into the distillation column. As a result the distillation column is subjected to reduced gas-hydraulic load and may therefore be operated with a smaller diameter. In addition the energy stored as heat in the reaction output is utilized for partial evaporation in the decompression into the flash vessel.

The flash vessel is additionally a separation stage and should be preferably configured to avoid an excessive gas velocity. Excessive gas velocity would be expected to result in increased droplet entrainment and more liquid catalyst-containing phase would pass into the condenser/into the phase separator. The flash vessels are generally operated at f-factors below 2. It is advantageous when the flash vessel is fitted with a suitable introduction apparatus for the liquid phase which allows simple separation of the gas phase and a good wetting ideally of the entire surface above the fill level with liquid. This reduces the formation of deposits at dry sites above the liquid-filled portion of the flash vessel. A further option is that of fitting the flash vessel with a spray apparatus that sprays the wall above the liquid phase and likewise allows wetting of the wall with liquid product.

It is advantageous when the upper portion of the flash vessel is fitted with a demister. The demister may be suitable wire meshes, though other embodiments having a droplet separating effect are also possible. This demister serves to separate and collect fine droplets. The demister should be mounted at a suitable distance from the level of the liquid. Advantageously, the demister may also be sprayed from above and below with solution containing a polymerization inhibitor (stabilizer) such as TEMPOL.

Irrespective of the configuration of the invention in other respects it has proven advantageous when between distillation column I and distillation column II a reverse osmosis is passed through. The reverse osmosis makes it possible to remove water from the bottoms phase from distillation column I. Such water is particularly low in organic constituents and may therefore relatively easily be sent for disposal, in particular to a biological treatment.

A reverse osmosis upstream of distillation stage II especially serves to save energy for evaporation of water since the amount withdrawn as permeate in the context of the reverse osmosis need no longer be evaporated. This is of economic interest especially in the case of high energy costs. The outflow from distillation stage I contains amine salts, formaldehyde, high-boilers and especially water. The amine salt has a particularly high retention and may therefore be retained in the retentate. Employable membranes include customary commercially available modules. The reverse osmosis may also be operated in multi-stage fashion. The modules are generally constructed with recycling of the retentate. Customary pressures in the stage having the highest retentate concentration are about 80 to 120 bar. The stage having the lowest retentate concentration is generally operated at a pressure of 20 to 50 bar. Initially the outflow of distillation stage I must be cooled to temperatures of 20° C. to 40° C. since reverse osmosis plants are generally operated in this temperature range. The retentate of the first stage exiting the reverse osmosis may be used in this case as cooling medium for energy integration. Further coolers may additionally employ cooling water or cooling brine.

### BRIEF DESCRIPTION OF THE DRAWINGS

FIG. 1 shows an inventive embodiment.

FIG. 2 shows an embodiment according to a comparative example.

FIG. 3 shows a preferred embodiment.

FIG. 4 shows a second preferred embodiment.

FIG. 5 shows a third preferred embodiment.

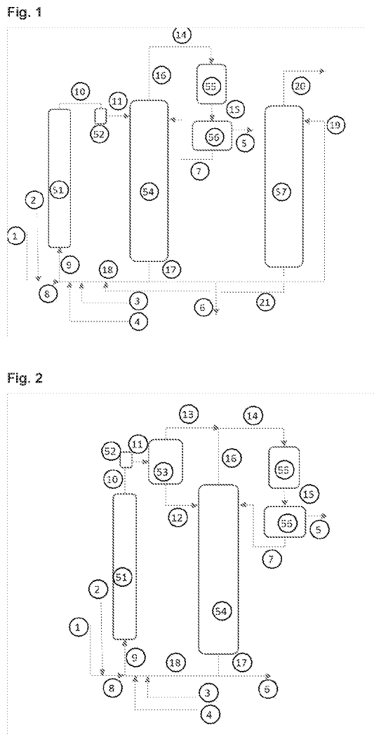

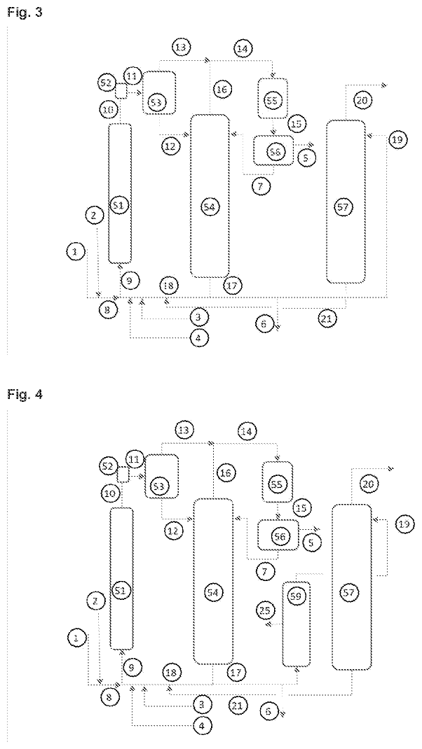

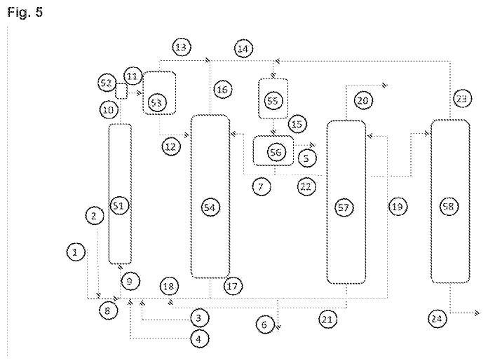

In terms of further processing of the permeate from reverse osmosis there are two different alternatives as advantageous embodiments:

In a first alternative the permeate of the reverse osmosis, optionally together with a portion of the condensate from distillation column II, is passed into the top of a distillation column IV. This distillation column IV removes a low-boiler from an MMA-containing fraction.

In a second alternative the permeate of the reverse osmosis, optionally together with a portion of the condensate from distillation column II, may be passed into a reactor II. This reactor II effects thermal cleavage of acetals. This reactor II may to this end be implemented in various forms. For example it is quite possible for reactor II to be a distillation column operated at corresponding temperatures in the column bottom. In such a case there may quite possibly be overlap with the first alternative or other embodiments of the present invention. For this embodiment it is crucial that temperatures and residence times that allow thermal cleavage of the acetals to a relevant extent are present in the corresponding plant component.

A modification of the present invention that has likewise proven very advantageous is one where the methacrolein-rich gas phase obtained in the flash vessel is condensed together with the gas phase generated in distillation column I and separated into an aqueous phase and an organic phase in phase separator I.

In a further preferred embodiment which may be performed together with or else independently of the other described embodiments the aqueous phase obtained in phase separator I is recycled into the distillation column I.

Alternatively, the aqueous phase obtained in phase separator I may then particularly preferably be passed wholly or partially into a distillation column I and the other portion of this phase optionally passed into distillation column III. In this alternative of passing into a distillation column III the aqueous phase is therein separated into a methacrolein-rich gas phase and a methacrolein-poor liquid phase. In particular the resulting methacrolein-rich gas phase is subsequently passed into condenser I.

It is very particularly preferable when the condensation of the methacrolein-rich gas phase of distillation column III is carried out in condenser I together with the methacrolein-rich gas phase from the flash vessel and/or the methacrolein-rich gas phase of distillation column I. In addition, two of these phases or all three may be combined beforehand and passed into the flash vessel together or separately from one another.

Distillation column III may be a column comprising random packings, structured packings or trays of any desired design. Different operating modes of this column are conceivable. The feed is for example introduced to the top of the column above the packing, the gas stream is withdrawn at the column top and the aqueous bottoms phase withdrawn at the column bottom. The gas stream is supplied directly to condenser I. This is advantageous since this makes it possible to recover residual amounts of methacrolein in the feed.

The column may be operated such that the feed of the column is run into the middle of the column and a liquid reflux is recycled at the column top above the packing. However, this presupposes a partial or complete condensation of the tops product in a dedicated condenser.

It is moreover advantageous to pass the methacrolein-poor liquid phase from distillation column III into an aerobic biological wastewater treatment. This methacrolein-poor liquid phase has such a low level of contamination that anaerobic biological wastewater treatment is the preferred method of disposal for this stream.

This makes it possible to reduce the amounts of wastewater to be worked up/the amount of fresh water to be added over the overall process.

This further has the advantage that in the oxidative esterification the small amounts of methacrolein still present may be reacted with an alcohol to afford a methacrylic ester, in particular with methanol to afford MMA. In particular the process according to the invention is especially used to convert the obtained methacrolein into a methacrylic ester, in particular into MMA, in a subsequent typically jointly continuously operated oxidative esterification (DOE). To this end the methacrolein obtained in the organic phase in phase separator I is generally passed into the reactor for this DOE step and therein wholly or partially converted into methyl methacrylate for example. The introduction of this organic phase from phase separator I into the DOE reactor may be carried out directly or indirectly—i.e. with passage through further purification steps.

It has further proven advantageous when the pH of the aqueous phase in phase separator I is lower than the pH of the condensed gaseous tops stream from distillation column II.

It is moreover advantageous when the pH of the condensed gas phase of distillation column III is lower than the pH of the condensed gaseous tops stream of distillation column II.

Both distillation column I and distillation column III should be operated such that the liquid phase in phase separator I has a pH of markedly below 7. At this pH amines are then bound as salts and dissolve in the organic methacrolein phase only to a very small extent which can have undesired adverse effects on the subsequent process step of direct oxidative esterification (DOE), in particular on the catalyst used in the DOE. Such effects result in poorer selectivity and reduced activity of the catalyst. In addition, a pH above 7 in the aqueous phase of phase separator I results in undesirable reactions in the alkaline aqueous phase comprising the dissolved methacrolein. This can also occur at the phase interface with the organic phase. This would especially comprise formation of turbidity or a relatively stable whitish layer between the organic phase and the aqueous phase. This so-called crud layer can be very stable and thus adversely affect reliable determination of the position of the phase interface to such an extent that controlling the aqueous reflux/feed to distillation column I or to distillation column III would no longer be possible. Cleaning shutdowns entailing considerable costs and losses would result. Distillation column II should therefore be operated such that the condensed gas phase is rather alkaline since a substantial task of this column is the separation of TMA.

### LIST OF REFERENCE SYMBOLS

### Streams

1 Propionaldehyde feed
2 Formaldehyde feed
3 Acetic acid feed
4 Dimethylamine feed
5 Methacrolein product stream
6 Wastewater
7 Aqueous reflux to distillation column I from phase separator I
8 Mixture of propionaldehyde and formaldehyde
9 Reactor feed
10 Reactor outflow
11 Pressure maintenance means outflow
12 Aqueous feed from flash vessel to distillation column I
13 Gaseous feed from flash vessel to condenser I
14 Gaseous feed from flash vessel and distillation column I to condenser I
15 Liquid outflow from condenser I to phase separator I
16 Distillation column I gaseous outflow
17 Distillation column I liquid outflow
18 Liquid recycling stream from distillation column I to reactor I
19 Aqueous feed to distillation column II
20 Gaseous outflow at top of distillation column II
21 Bottoms outflow from distillation column II to reactor I
22 Aqueous feed to distillation column III from phase separator I
23 Gaseous outflow from distillation column III to condenser I
24 Bottoms outflow from distillation column III
25 Permeate reverse osmosis plant

### Apparatuses

51 Reactor I
52 Pressure maintenance means
53 Flash vessel
54 Distillation column I
55 Condenser I
56 Phase Separator I
57 Distillation Column II
58 Distillation column III
59 Reverse osmosis plant I Various apparatuses which have nothing directly to do with the invention per se but would be implemented by a person skilled in the art as standard are not reproduced in the figures for the sake of clarity. This applies, for example, to pumps or heat exchangers other than condenser I.

### EXAMPLES

### 1 Comparison Example

54 kg/h of propionaldehyde (Oxea Oberhausen) and 51 kg/h of formaldehyde (55%) were mixed in a static mixer and preheated to a temperature of about 130° C. in an oil-heated coil heater. About 85 kg/h of bottoms outflow of the methacrolein workup column are mixed with 1.53 kg/h of acetic acid and 2.59 kg/h of dimethylamine (40%) and the obtained catalyst mixture was likewise preheated to 130° C. in an oil-heated pipe coil. Subsequently, in a further static mixer the aldehyde solution and the catalyst mixture were mixed and this mixture was passed into the oil-heated tubular reactor having a diameter of 10 mm and a length of about 8 m. The oil flow temperature was 160° C. The pressure in the reactor was adjusted to 30 bar using a valve arranged immediately downstream of the reactor. The reaction output was decompressed into a flash vessel at a temperature of about 167° C. The temperature in the flash vessel was about 83° C. The liquid phase was passed to the top of the methacrolein workup column having a Sulzer Melapak packing (ID=100 mm, length=6.4 m). The gas phase from this column was combined with the gas phase from the flash vessel and condensed and the condensate was fed into a decanter. About 66 kg/h of methacrolein in a purity of 96.5% were obtained. The aqueous phase obtained in the decanter was recycled to the top of the column at a mass flow of 30 kg/h. The bottom of the column was heated with 10 bar steam using forced circulation (circulation about 600 kg/h) and a shell and tube heat exchanger.

The column was operated at standard pressure. About 44 kg/h of the bottoms outflow were discharged as wastewater and about 85 kg/h of the same aqueous bottoms outflow were recycled to the reactor as recycle. 0.025 mol of dimethylamine per mol of propionaldehyde and 0.027 mol of acetic acid per mol of propionaldehyde were employed as feed to the plant. The molar ratio of formaldehyde to propionaldehyde in the feed to the plant was approximately 0.985. The molar MAL yield was about 98.5%.

### 2 Distillation Experiments with Wastewater from Methacrolein Synthesis

Wastewater from the production of methacrolein according to a composition as in comparative example 1 was distilled in a column (DN 100, Melapack 6.4 m in length). 30 kg/h of wastewater were added to the top of the column and the obtained gas phase was condensed at about 20° C. and collected in a distillation receiver. The bottom of the column was heated with 10 bar steam using forced circulation and a heat exchanger. The bottoms output was withdrawn from the bottoms under fill level control. The temperature was determined at various points of the column, in particular in the bottom and in the top of the column. The pressure was determined in the column top and in the column bottom. The distillation column was operated at standard pressure. The amount of steam applied was adjusted according to the desired degree of concentration. The degree of concentration indicates the ratio of the feed stream to the bottoms stream. The degree of concentration parameter may be elucidated with reference to the following example: At a feed stream of 30 kg/h, a distillate stream of 27 kg/h and a bottoms stream of 3 kg/h the degree of concentration, for example, is 10.

Trimethylamine (TMA), dimethylamine (DMA), acetic acid (ACA), propionic acid (PRA), water and high-boilers (HB) were determined in the feed, the bottoms stream and the distillate. Based on the particular mass flows and the analyses thereof the yield for each component as well as for the bottoms stream and for the distillate were determined. The distillation yield is the molar flow of a component in the distillate based on the feed stream of the component to the column multiplied by 100. The yield in the bottoms is the molar flow of a component in the bottoms stream based on the feed stream of the component to the column multiplied by 100. The yields were normalized to the feed stream of the respective component.

The pH was additionally measured in all streams (feed stream, bottoms stream, distillate).

The accompanying table summarizes the results of the distillation experiments.

| | | Bottoms product | | | | | | | | Distillates | | | | | |
|---|---|---|---|---|---|---|---|---|---|---|---|---|---|---|---|
| | | | H2O | | Y DMA | Y TMA | Y ACA | Y PRA | Y H8 | | Y DMA | Y TMA | Y ACA | Y PRA | Y H8 |
| | | T | [% by | | [% by | [% by | [% by | [% by | [% by | | [% by | [% by | [% by | [% by | [% by |
| No. | DoC | [° C.] | mass] | pH | mass] | mass] | mass] | mass] | mass] | pH | mass] | mass] | mass] | mass] | mass] |
| 2.1 | 1.2 | 100 | 88.8 | 6.18 | 100 | 96.2 | 99.0 | 96.8 | 98.3 | 4.95 | 0.10 | 3.84 | 0.96 | 3.21 | 1.7 |
| 2.2 | 1.2 | 100 | 88.1 | 6.26 | 100 | 96.2 | 99.1 | 97.5 | 99.1 | 4.97 | 0.10 | 3.78 | 0.93 | 2.50 | 0.9 |
| 2.3 | 1.2 | 99 | 88.6 | 6.19 | 100 | 96.1 | 99.0 | 97.7 | 99.2 | 4.95 | 0.17 | 3.92 | 1.00 | 2.25 | 0.8 |
| 2.4 | 2.0 | 100 | 84.0 | 5.76 | 100 | 91.4 | 97.5 | 92.2 | 76.3 | 4.75 | 0.01 | 8.60 | 2.47 | 7.82 | 23.7 |
| 2.5 | 2.3 | 101 | 81.2 | 5.75 | 100 | 97.2 | 100.0 | 99.0 | 76.6 | 9.59 | 0.03 | 2.8 | — | 1.00 | 23.4 |
| 2.6 | 2.3 | 101 | 81.5 | 5.75 | 100 | 97.3 | 100.0 | 99.4 | 77.0 | 9.56 | 0.03 | 2.7 | — | 0.64 | 23.0 |
| 2.7 | 3.3 | 102 | 73.7 | 5.86 | 100 | 88.0 | 100.0 | 99.3 | 70.4 | 9.04 | 0.09 | 12.0 | — | 0.70 | 29.5 |
| 2.8 | 3.2 | 102 | 75.2 | 5.86 | 108 | 78.9 | 97.1 | 97.6 | 72.7 | — | 0.04 | 21.1 | 2.91 | 7.35 | 27.3 |
| 2.9 | 5.5 | 105 | 59.9 | 6.08 | 100 | 50.3 | 98.3 | 95.2 | 58.9 | — | 0.22 | 49.7 | 1.68 | 4.82 | — |
| 2.10 | 5.5 | 105 | 59.8 | 6.08 | 100 | 49.4 | 98.4 | 95.4 | 62.7 | 8.79 | 0.18 | 50.5 | 1.64 | 4.65 | — |
| 2.11 | 8.3 | 108 | 43.1 | 6.09 | 96.9 | 9.3 | 96.8 | 91.1 | 62.6 | 9.34 | 3.1 | 90.7 | 3.2 | 8.9 | 37.4 |
| 2.12 | 9.3 | 109 | 41.6 | 6.05 | 96.7 | 6.2 | 96.6 | 91.2 | 53.0 | 9.44 | 3.3 | 93.8 | 3.4 | 8.8 | 42.0 |
| 2.13 | 9.4 | 111 | 38.9 | 6.41 | 95.4 | 0.7 | 97.6 | 94.6 | 65.9 | 10.19 | 4.6 | 99.3 | 2.4 | 5.4 | 33.1 |
| 2.14 | 9.3 | 110 | 38.7 | 6.43 | 94.5 | 0.6 | 97.7 | 95.6 | 67.8 | 10.31 | 5.5 | 99.4 | 2.3 | 4.4 | 32.2 |
| 2.15 | 12.0 | 115 | 32.5 | 6.26 | 87.4 | 0.7 | 97.2 | 96.2 | 45.4 | 9.30 | 12.6 | 99.3 | 2.8 | 3.8 | 54.5 |
| 2.16 | 12.0 | 117 | 31.4 | 6.25 | 87.0 | 0.3 | 98.4 | — | 40.2 | 9.42 | 13.0 | 99.7 | 1.6 | — | 59.3 |
| 2.17 | 12.0 | 117 | 27.8 | 6.41 | 88.0 | 0.3 | 98.5 | — | 49.4 | 9.39 | 12.0 | 99.7 | 1.5 | — | 50.6 |
| 2.18 | 12.0 | 117 | 27.8 | 6.41 | 88.0 | 0.3 | 98.5 | — | 49.4 | 9.39 | 12.0 | 99.7 | 1.5 | — | 50.6 |

In the context of the present description % by mass and % by weight are to be understood as meaning identical parameters.

Distillation experiments 2.1 to 2.4 were carried out at relatively low degrees of concentration from 1.2 to 2.0. Accordingly the water content in the bottoms product was between 84% by weight and 89% by weight and was still relatively high compared to the other experiments. The distillate showed a pH of about 5 and only relatively small amounts of trimethylamine were separable overhead. Dimethylamine could hardly be found in the distillate at all. Some free acetic acid was found in the distillate, thus resulting in the low pH. The retention of propionic acid in the bottoms is greater than 90%. The bottoms had a pH of about 6 and were therefore in the acidic range.

Distillation experiments 2.5 to 2.10 were performed at degrees of concentration of 2.3 to 5.5 and bottoms products having water contents of 60% by weight to about 81% by weight were obtained. According to the concentration methods discussed in the prior art the hitherto described water content of concentrates obtained in evaporative concentration of aqueous solutions from methacrolein synthesis is about 60% by weight. Up to this water content it remained possible to achieve a very good retention of DMA in the bottoms, i.e. to obtain a very high bottoms yield for DMA, which runs contrary to the prior art teaching of the volatility of DMA and is thus exceptionally surprising.

Surprisingly, up to 50% of the TMA employed in the feed was able to be separated. This is very surprising on account of the teaching of the prior art since TMA and DMA exhibit only a relatively small boiling point difference of about 4° C. Standard pressure DMA boils at 7° C. while TMA has a boiling point of 2.9° C. However there remained a considerable amount of TMA in the bottoms product so that a considerable amount of acetic acid remains bound as TMA acetate. The distillate surprisingly exhibited a pH of 8.8 to 9.6 and the distillate was accordingly an alkaline solution. The bottoms had a pH of about 6 and were in the acidic range. The retention of acetic acid and propionic acid was greater than 95% and greater than 90%, respectively.

Distillation experiments 2.11 to 2.14 were performed at degrees of concentration of 8.3 to 9.4 and bottoms products having water contents between 39% by weight and 43% by weight were obtained. These water contents were markedly lower than the water content of 60% by weight in concentrates obtained in the distillation of aqueous catalyst-containing solutions from the synthesis of methacrolein as described in the prior art. The retention of DMA in the bottoms (yield in the bottoms) remained at 95% or greater than 95%. TMA was surprisingly able to be separated to an extent of 90% or to an extent of 99% and there was accordingly more free acetic acid in the bottoms. The yield of acetic acid in the distillate remained relatively low and was on average about 2.7%. It remained possible to retain propionic acid in the bottoms to an extent of >91%. In addition, about 32% to 42% of high-boilers were able to be separated by the distillate. The distillate exhibited a pH of 9.3 to 10.3 and was alkaline. The bottoms had a pH of about 6 and were in the acidic range.

In distillation tests 2.15 to 2.18, the degree of concentration was increased again and bottoms product having water contents of around 30% by weight were obtained. At these relatively low water contents an increasing amount of DMA was lost via the gas phase and the DMA yield in the tops fraction was markedly greater than 10%. TMA was completely distilled out of the bottoms product. These experiments were also able to achieve a high separation of high-boilers of about 50% to 60%. The bottoms product yields of acetic acid and propionic acid remained in a good range of above 90%. As in all previously described examples the bottoms had a pH of about 6.4 and were in the acidic range. The distillate of these experiments was alkaline with a pH of about 9.4.

### 3 Combination of Catalyst Distillation and Methacrolein Synthesis

Comparative example 3.1: 1717 g/h propionaldehyde are reacted with 1604 g/h or 1595 g/h of 55% formaldehyde. The propionaldehyde employed contains about 0.2% by weight of propionic acid. Without operation of the catalyst distillation 81 g/h of DMA (40% by weight) and 59.4 g/h of acetic acid (80% by weight) are required. The reactor is operated at a water content in the feed of 56.3%. A reactor feed ratio of 0.075 mol of DMA per mol of propionaldehyde is established. The reactor is operated at about 160° C. and provides a residence time of about 10 sec. A ⅛ inch stainless steel capillary in a heated oil bath is used. The reactor product is decompressed in a flash vessel and the liquid flash product is supplied to a methacrolein workup column. This random-packed column has a diameter of 50 mm and a length of 1.5 m. The gas phase from the flash vessel and the gaseous product from the methacrolein workup column is supplied to a condenser. The condensate is separated into two phases in a decanter. About 2.05 kg/h of methacrolein is obtained at the top of the methacrolein workup column, thus corresponding to a yield of about 98.3%. The aqueous phase is returned to the methacrolein workup column. The liquid recycling stream to the reactor from the bottom of the methacrolein workup column is about 3220 g/h. About 1413 g/h of wastewater are obtained at the column bottom and collected in a receiver.

In examples 3.2 to 3.5 a catalyst distillation column is added (50 mm diameter, Raschig rings, 1.5 m in length). This column is supplied with 90% of the bottoms output from the methacrolein workup column at the column top of the packing of Raschig rings. The remaining 10% of the bottoms output of the methacrolein workup column is discharged as wastewater and collected in the bottoms receiver. The distillate of the catalyst distillation column is condensed and collected in the distillate receiver. The bottoms product of the catalyst distillation column is recycled to the reactor. The catalyst distillation column is heated via a heating plug at the column bottom. Heating power is adjusted according to the desired degree of concentration. The recycle stream from the bottom of the methacrolein column, the DMA feed and the acetic acid feed are adjusted according to the desired parameters (DMA/PA, acid/amine ratio and water content to the reactor). The results are shown in the table. The desired methacrolein yield of about 98.3% is achieved in each case.

| | | Example | | | | |
|---|---|---|---|---|---|---|
| Stream | unit | 3.1 | 3.2 | 3.3 | 3.4 | 3.5 |
| Propionaldehyde | [g/h] | 1717 | 1717 | 1717 | 1717 | 1717 |
| Formalin - 55% | [g/h] | 1604 | 1595 | 1595 | 1595 | 1595 |
| Dimethylamine (40%) | [g/h] | 81.0 | 24.1 | 17.5 | 18.7 | 24.8 |
| Acetic acid (80%) | [g/h] | 59.4 | 16.5 | 6.7 | 5.7 | 5.5 |
| Recycle | [g/h] | 3220 | 1917 | 3398 | 3440 | 3365 |
| MAL column feed | [g/h] | 3724 | 3746 | 3753 | 3752 | 3751 |
| MAL column reflux | [g/h] | 819 | 824 | 826 | 825 | 825 |
| Methacrolein | [g/h] | 2048 | 2060 | 2064 | 2064 | 2063 |
| MAL column bottoms product | [g/h] | 4633 | 4954 | 5037 | 4919 | 4787 |
| Wastewater | [g/h] | 1413 | 304 | 164 | 148 | 142 |
| Catalyst distillation gas phase | [g/h] | — | 1003 | 1123 | 1139 | 1152 |
| Catalyst distillation | [g/h] | — | 1730 | 353 | 191 | 128 |
| bottoms product | | | | | | |
| MAL column bottoms product | [% by | 91.5 | 89 | 89 | 89 | 90 |
| water content | mass] | | | | | |
| Cat distillation bottoms | [% by | — | 84 | 61 | 44 | 32 |
| product water content | mass] | | | | | |
| Difference | [% by | — | 5 | 28 | 46 | 59 |
| | mass] | | | | | |
| Reactor feed water content | [% by | 56.3 | 56.3 | 56.3 | 56.3 | 56.3 |
| | mass] | | | | | |
| DMA/PA reactor feed | mol/mol | 0.075 | 0.075 | 0.075 | 0.075 | 0.075 |
| Acids/amines reactor feed | mol/mol | 1.16 | 1.17 | 1.16 | 1.16 | 1.17 |
| Decanter aq. phase pH | | 5.5 | 5.4 | 5.5 | 5.4 | 5.6 |
| Cat distillation distillate pH | | — | 5 | 9.1 | 9.0 | 9.2 |
| DMA addition saving | % | — | 70.3 | 78.4 | 77.0 | 69.4 |
| Acetic acid addition saving | % | — | 72.2 | 88.8 | 90.5 | 90.7 |

Example 3.1 is an example without catalyst distillation. The water content in the bottoms of the methacrolein workup column is 91.5% by weight.

In examples 3.2 to 3.5 the methacrolein synthesis is operated with a catalyst distillation. The water content in the bottoms of the catalyst distillation is varied. The water content in the bottoms of the catalyst distillation is 84% by weight (example 3.2), 61% by weight (example 3.3), 44% by weight (example 3.4) and 32% by weight (example 3.5).

At excessively high water contents of about 84% by weight in the bottoms of the catalyst distillation/at a relatively low difference between the water content in the bottoms product of the methacrolein workup column and the water content in the bottoms of the catalyst distillation of 5% by weight, about 70% acetic acid and DMA can be saved in example 3.2. At a water content of 84% by weight in the bottoms of the catalyst distillation the distillate remains weakly acidic and could come into contact with methacrolein without adverse effects.

In example 3.3 the difference between the water content of the bottoms of the methacrolein workup column and the water content in the bottoms of the catalyst distillation is 28% by weight. This example allows savings of about 78.4% of DMA and 88.8% of acetic acid relative to comparative example 3.1 and this constitutes a considerable saving. The water content in the bottoms of the catalyst distillation is 61% by weight and an alkaline distillate having a pH of about 9 is obtained in the catalyst distillation column.

In example 3.4 the difference between the water content of the bottoms of the methacrolein workup column and the water content in the bottoms of the catalyst distillation is about 46% by weight. This example likewise allows considerable savings of catalyst of about 77% of DMA and about 90.5% of acetic acid relative to comparative example 3.1. The water content in the bottoms of the catalyst distillation is 44% by weight and an alkaline distillate having a pH of about 9 is obtained.

In example 3.5 the difference between the water content of the bottoms product from the methacrolein workup column and the water content in the bottoms of the catalyst distillation is about 59% by weight. This example likewise allows considerable savings of catalyst of about 69.4% of DMA and 90.4% of acetic acid relative to comparative example 3.1. However the saving of catalyst is slightly reduced relative to example 3.4.

The water content in the bottoms of the catalyst distillation is 32% by weight and an alkaline distillate having a pH of about 9 is obtained. At excessively high concentrations DMA is discharged into the top of the catalyst distillation to a markedly greater extent.

### 4 Combination of Catalyst Distillation Column with Sidestream Column in Methacrolein Synthesis

The plant consisting of methacrolein synthesis and catalyst distillation is augmented with a column for distillation of a portion of the aqueous phase from the decanter. This column, which corresponds to distillation column III according to the invention and in the following is also referred to as a sidestream column, is especially intended to strip out residual amounts of methacrolein (about 5%) from the aqueous phase of the decanter. The other portion of the aqueous phase, which is obtained in the decanter, is further supplied to the methacrolein workup column.

The gaseous tops stream of this column is supplied to the condenser into which the gaseous streams of the methacrolein workup column and the flash vessel are also introduced. The aqueous bottoms stream of distillation column III is collected in a bottoms receiver. The following table shows the examples (4.1 to 4.3) of the combination of distillation column III with the catalyst distillation in the methacrolein synthesis. Examples 3.1 and 3.4 are shown for comparison.

| | | Example | | | | |
|---|---|---|---|---|---|---|
| Stream | unit | 3.1 | 3.4 | 4.1 | 4.2 | 4.3 |
| Propionaldehyde | [g/h] | 1717 | 1717 | 1717 | 1717 | 1717 |
| Formalin - 55% | [g/h] | 1604 | 1595 | 1595 | 1595 | 1595 |
| Dimethylamine (40%) | [g/h] | 81.0 | 18.7 | 17.2 | 16.2 | 13.6 |
| Acetic acid (80%) | [g/h] | 59.4 | 5.7 | 5.0 | 4.8 | 3.6 |
| Recycle | [g/h] | 3220 | 3440 | 3574 | 3699 | 4227 |
| MAL column feed | [g/h] | 3724 | 3752 | 3730 | 3732 | 3739 |
| MAL column reflux | [g/h] | 819 | 825 | 821 | 821 | 823 |
| Methacrolein | [g/h] | 2048 | 2064 | 2051 | 2052 | 2057 |
| Sidestream column bottoms | [g/h] | — | — | 253 | 421 | 760 |
| MAL column bottoms product | [g/h] | 4633 | 4919 | 4778 | 4719 | 4864 |
| Wastewater | [g/h] | 1413 | 148 | 120 | 102 | 64 |
| Catalyst distillation gas phase | [g/h] | — | 1139 | 911 | 758 | 450 |
| Catalyst distillation | [g/h] | — | 191 | 173 | 159 | 123 |
| bottoms product | | | | | | |
| MAL column bottoms product | [% by | 91.5 | 89 | 88 | 87 | 83 |
| water content | mass] | | | | | |
| Cat distillation bottoms | [% by | — | 44 | 44 | 44 | 44 |
| product water content | mass] | | | | | |
| Difference | [% by | — | 46 | 44 | 43 | 40 |
| | mass] | | | | | |
| Reactor feed water content | [% by | 56.3 | 56.3 | 56.3 | 56.3 | 56.3 |
| | mass] | | | | | |
| DMA/PA reactor feed | mol/mol | 0.075 | 0.075 | 0.075 | 0.075 | 0.075 |
| Acids/amines reactor feed | mol/mol | 1.16 | 1.16 | 1.16 | 1.17 | 1.16 |
| Decanter aq phase pH | | 5.5 | 5.4 | 5.5 | 5.4 | 9.0 |
| Cat distillation distillate pH | | — | 9 | 9.1 | 9.0 | 9.2 |
| DMA addition saving | % | — | 77.0 | 78.8 | 80.0 | 83.2 |
| Acetic acid addition saving | % | — | 90.5 | 91.6 | 92.0 | 94.0 |
| Catalyst distillation steam | % | — | — | 13.4 | 22.3 | 40.5 |
| demand saving | | | | | | |

Example 3.1 is the comparative example without catalyst distillation and example 3.4 combines a catalyst distillation with the methacrolein synthesis and a water content in the bottoms of the catalyst distillation of about 44% by weight is achieved. Examples 4.1 to example 4.3 show examples in which a distillation column III, in which the aqueous phase from the decanter is partially worked up separately, is combined with a methacrolein synthesis with catalyst distillation. Different amounts of sidestream are obtained in these examples.

In example 4.1 about 253 g/h are obtained at the bottom of distillation column III and compared to example 3.4 the water content in the bottoms of the methacrolein workup column is lower than example 3.4 at about 88% by weight. The increased concentration in the bottoms of the methacrolein workup column reduces the gaseous stream obtained in the catalyst distillation. Since about 1 ton of steam is required per ton of gaseous stream from this stage this equates to a steam saving of around 13.4% relative to example 4.1. This includes taking into account the steam consumption of distillation column III. The DMA saving is about 78.8% and the saving of acetic acid is 91.6%. Thus, in comparison to example 3.4, the use of distillation column III can further reduce catalyst consumption while achieving a marked saving of steam. At the water content in the bottoms of the methacrolein workup column of 88% by weight established in example 4.1 the aqueous phase in the decanter remains slightly acidic at a pH of about 5.5.

In example 4.2 about 421 g/h are obtained at the bottom of distillation column III and compared to example 3.4 the water content in the bottoms of the methacrolein workup column is slightly lower than example 3.4 at about 87% by weight. The increased concentration in the bottom of the methacrolein workup column considerably reduces the gaseous stream obtained in the catalyst distillation. Since about 1 ton of steam is required per ton of gaseous stream from this stage this equates to a steam saving of around 22.3% relative to example 4.1 including the steam amount supplied to distillation column III. The DMA saving is about 80% and the saving of acetic acid is 92%. Thus, in comparison to example 3.4, the use of distillation column III can further reduce catalyst usage while achieving a marked reduced steam consumption. At the water content in the bottoms of the methacrolein workup column of 87% by weight established in example 4.2 the aqueous phase in the decanter remains slightly acidic at a pH of about 5.4.

In example 4.3 the bottoms stream of distillation column III is further increased and about 760 g/h are withdrawn at the bottom of distillation column III. This increases the concentration in the bottom of the methacrolein workup column and the water content in the bottoms of this column decreases to a value of 83% by weight. This leads to an increased output of TMA and an alkaline aqueous phase is obtained in the methacrolein decanter. The pH is about 9. The alkaline distillate may result in markedly increased occurrence of polymeric deposits in the decanter and also in increased occurrence of whitish deposits (so-called crud layer) at the phase interface between the methacrolein and the aqueous phase. In long-term operation of the plant these deposits would be expected to cause increased problems. While the relatively high degree of concentration in the methacrolein workup stage makes it possible to achieve a high steam saving of 40.5% and increased DMA and acetic acid savings of 83.2% and 94%, the increased polymeric deposits would be expected to result in a markedly reduced availability of the plant due to a very severe increase in cleaning shutdowns.

1: A process for continuously performing a Mannich reaction in which methacrolein is produced in reactor I from formaldehyde and propionaldehyde with at least one acid and dimethylamine as catalysts, the process comprising:
passing an output from reactor I directly or indirectly into distillation column I in which a methacrolein-containing low-boiling phase is separated from an aqueous high-boiling phase having a water content of greater than 85% by weight,
wherein that the aqueous high-boiling phase is partially directly or indirectly passed into distillation column II from which a gaseous tops stream is directly or indirectly, wholly or partially, sent to a thermal oxidizer and a liquid bottoms phase is wholly or partially recycled into reactor I with a water content which is at least 20% by weight lower relative to the high-boiling aqueous phase from distillation column I.

2: The process according to claim 1, wherein the methacrolein-containing low-boiling phase from distillation column I is condensed in condenser I connected downstream of distillation column I and the condensate from this condenser I is separated into a liquid aqueous methacrolein-poor phase and a liquid methacrolein-rich phase in a downstream phase separator I.

3: The process according to claim 1, wherein the reactor output from reactor I is first decompressed and passed into a flash vessel, in this flash vessel a methacrolein-rich gas phase is separated from a high-boiling aqueous liquid phase and in that the liquid phase is passed into distillation column I.

4: The process according to claim 1, wherein between distillation column I and distillation column II a reverse osmosis is passed through, by which water is removed from the bottoms phase of distillation column I.

5: The process according to claim 4, wherein a permeate of the reverse osmosis, optionally together with a portion of the condensate from distillation column II, is passed into the top of distillation column IV, by which low-boilers are removed from an MMA-containing fraction.

6: The process according to claim 4, wherein the permeate of the reverse osmosis, optionally together with a portion of the condensate of distillation column II, is passed into reactor II in which acetals are thermally cleaved.

7: The process according to claim 3, wherein the methacrolein-rich gas phase obtained in the flash vessel is condensed together with the gas phase generated in distillation column I and separated into an aqueous phase and an organic phase in phase separator I.

8: The process according to claim 2, wherein the aqueous phase obtained in phase separator I is wholly or partially recycled into distillation column I and optionally partially passed into distillation column III where this aqueous phase is separated into a methacrolein-rich gas phase and a methacrolein-poor liquid phase, wherein the methacrolein-rich gas phase is subsequently passed into condenser I.

9: The process according to claim 8, wherein the methacrolein-poor liquid phase from distillation column III is supplied to an aerobic or anaerobic biological wastewater treatment or is directly or indirectly passed into a reactor for oxidative esterification of methacrolein.

10: The process according to claim 1, wherein compared to the high-boiling aqueous phase of distillation column I the liquid bottoms phase of distillation column II has a water content which is between 40% by weight and 55% by weight lower.

11: The process according to claim 2, wherein the pH of the aqueous phase in phase separator I is lower than the pH of the condensed gaseous tops stream from distillation column II.

12: The process according to claim 5, wherein the pH of the condensed gas phase of distillation column III is lower than the pH of the condensed gaseous tops stream of distillation column II.
